(19) United States
(12) Patent Application Publication
(10) Pub. No.: US 2024/0288433 A1
(43) Pub. Date: Aug. 29, 2024

(54) COMBINED METHOD OF DETECTING PROTEINS IN HUMAN SAMPLES AND MPMRI DATA

(71) Applicant: ProteoMedix AG, Schlieren (CH)

(72) Inventors: Ralph SCHIESS, Zürich (CH); Christian BRÜHLMANN, Zürich (CH); Oliver STRAUB, Bern (CH); Alcibiade ATHANASIOU, Dättwil (CH)

(73) Assignee: ProteoMedix AG, Schlieren (CH)

(21) Appl. No.: 18/570,192

(22) PCT Filed: Jun. 16, 2022
(86) PCT No.: PCT/EP2022/066453

§ 371 Date: Dec. 14, 2023

(30) Foreign Application Priority Data
Jun. 29, 2021 (CH) 00751/21

(51) Int. Cl. G01N 33/574

(52) U.S. Cl. CPC G01N 33/57434; G01N 2470/04

(57) ABSTRACT

Method for collecting information about the health status of a subject involving the quantitative detection, in serum, plasma or blood of the subject, of the concentration of THBS1, as well as the proportion of free PSA (% fPSA) in combination with data obtained from multi-parametric prostate magnetic resonance imaging data.

### TECHNICAL FIELD

The present invention relates to the field of methods for the measurement of proteins and other parameters in human samples, in particular in human serum, plasma or blood, and it also relates to assays and uses of such assays, in particular for risk assessment.

### PRIOR ART

The early detection of clinically significant prostate cancer (ISUP Group Grade ≥2, csPCa) decreases prostate cancer mortality. The classic approach for early detection of prostate cancer (PCa), based on serum prostate-specific antigen (PSA), digital rectal examination (DRE), and systematic biopsies, has been fairly disapproved due to the high rates of unnecessary biopsies and overdetection of insignificant PCa (iPCa).

The recent improvement of early detection of csPCa has come from multiparametric magnetic resonance imaging (mpMRI) and guided biopsies using MRI as a template.

Nevertheless, the efficacy of this new strategy leaves room for improvement with a more accurate selection of candidates for prostate biopsy, especially when low risk of csPCa exists. Prostate Imaging Reporting and Data System (PI-RADS) scores range from 1-5 and a score of 3, often referred to as indeterminate mpMRI, defines a subset of men with low risk of csPCa, being its detection rate less than 20%. In this scenario, the use of PSA density (PSAD), some markers and predictive models, e.g. the ERSPC online risk calculator (RC), that include clinical variables and mpMRI has been proposed.

Steuber et al in European Urology Oncology, Volume 5, Issue 3, June 2022, Pages 321-327, disclose, that prostate-specific antigen (PSA)-based detection of prostate cancer (PCa) often leads to negative biopsy results or detection of clinically insignificant PCa, more frequently in the PSA range of 2-10 ng/ml, in men with increased prostate volume and normal digital rectal examination (DRE). The study evaluated the accuracy of Proclarix, a novel blood-based diagnostic test, to help in biopsy decision-making in this challenging patient population. Ten clinical sites prospectively enrolled 457 men presenting for prostate biopsy with PSA between 2 and 10 ng/ml, normal DRE, and prostate volume ≥35 cm3. Transrectal ultrasound-guided and multiparametric magnetic resonance imaging (mpMRI)-guided biopsy techniques were allowed. Serum samples were tested blindly at the end of the study. Diagnostic performance of Proclarix risk score was established in correlation to systematic biopsy outcome and its performance compared with % free PSA (% fPSA) and the European Randomised Study of Screening for Prostate Cancer (ERSPC) risk calculator (RC) as well as Proclarix density compared with PSA density in men undergoing mpMRI.

The sensitivity of Proclarix risk score for clinically significant PCa (csPCa) defined as grade group (GG) ≥2 was 91% (n=362), with higher specificity than both % fPSA (22% vs 14%; difference=8% [95% confidence interval {CI}, 2.6-14%], p=0.005) and RC (22% vs 15%; difference=7% [95% CI, 0.7-12%], p=0.028). In the subset of men undergoing mpMRI-fusion biopsy (n=121), the specificity of Proclarix risk score was significantly higher than PSA density (26% vs 8%; difference=18% [95% CI, 7-28%], p<0.001), and at equal sensitivity of 97%, Proclarix density had an even higher specificity of 33% [95% CI, 23-43%]. In a routine use setting, Proclarix accurately discriminated csPCa from no or insignificant PCa in the most challenging patients. Proclarix represents a valuable rule-out test in the diagnostic algorithm for PCa, alone or in combination with mpMRI.

Hayley et al in Journal of Clinical Oncology, Volume 38, Issue 6, p 278 disclose the use of multi-parametric magnetic resonance imaging (mpMRI) being a significant advance in the diagnosis of prostate cancer (PCa) recommended in a number of guidelines. There are considerable resource implications in scanning all men at risk of PCa. Furthermore, a significant number of mpMRIs are reported as indeterminate, leading to unnecessary biopsies. Proclarix is a CE-marked test based on two novel biomarkers, thrombospondin 1 (THBS1) and cathepsin D (CTSD), combined with PSA and age. A software algorithm returns a risk score that can be used as an aid in the identification of clinically significant PCa (any Grade Group 2 or greater). We aimed to assess the potential of Proclarix to identify those men who could safely avoid an upfront mpMRI or those men who could avoid biopsy when the mpMRI was indeterminate. Methods: Proclarix was correlated retrospectively with diagnostic data from 282 men recruited in the INNOVATE study (NCT02689271). INNOVATE involved men undergoing mpMRI followed by targeted and systematic biopsies in those with a suspicious mpMRI. Results: Median age and PSA were 66 (IQR 59-70) and 5.4 (3.8-7.8) ng/mL. 182 (65%) men underwent biopsy and 78 (43%) had GG≥2 PCa. Application of Proclarix in all 282 men undergoing mpMRI resulted in a sensitivity for clinically significant PCa (GG≥2) of 91%, a negative predictive value (NPV) of 92% and 38% specificity. When normalized to the same sensitivity of 91%, % fPSA resulted in both lower NPV (89%) and specificity (28%) when compared to Proclarix. 144 (51%) men had an indeterminate mpMRI of whom 84 (58%) had a biopsy and 13 (15%) had GG≥2 PCa. In these men, Proclarix had an NPV of 100%, at 100% sensitivity and a specificity of 34%. When results were compared using equal sensitivity, PSA density (cut-off 0.05 ng/ml), which is frequently used to inform the need for biopsy, had 10% specificity.

Conclusions: The use of Proclarix could potentially allow 38% of men to avoid undergoing an mpMRI. In men with an indeterminate mpMRI, Proclarix could allow one-third to safely avoid biopsies without missing any clinically significant cancer.

WO-A-2018011212 relates to a method for collecting information about the health status of a subject is proposed involving the quantitative detection, in serum, plasma or blood of the subject, of the concentration of THBS1, the proportion of free PSA (% fPSA), preferably including the concentration of at least one protein selected from the group consisting of CTSD, OLFM4, ICAM1.

Gröberg et al in Eur Urol. 2018; 74(6):722-728 assessed the performance of combining a blood-based biomarker panel and magnetic resonance imaging (MRI)-targeted biopsies for prostate cancer detection. They used a prospective, multicenter, paired diagnostic study design. A total of 532 men aged 45-74 yr referred for prostate cancer workup were included during 2016-2017. Participants underwent blood sampling for analysis of the Stockholm3 test including protein biomarkers, genetic polymorphisms, and clinical variables; 1.5 T MRI; systematic prostate biopsies; and MRI-targeted biopsies to lesions with Prostate Imaging Reporting and Data System version 2≥3. The main outcome was numbers of detected prostate cancer characterized by grade group (GG) and the number of performed biopsies using relative sensitivity (RS). Median prostate-specific antigen was 6.3 ng/ml, and mean age was 63.9 yr. Targeted and systematic biopsies detected 170 and 162 GG≥2 tumors, respectively (RS 1.05; 95% confidence interval [CI] 0.96-1.14). Compared with performing systematic biopsies on all men, performing targeted and systematic biopsies only on men with >10% risk of GG≥2 cancer, as predicted by the Stockholm3 test, required 62% (95% CI 58-66) of the biopsy procedures and detected 58% (95% CI 48-70) of GG 1 disease, with increased sensitivity for GG≥2 detection (RS 1.10; 95% CI 1.02-1.17). Performing only targeted biopsies in men with elevated Stockholm3 test altered these results only slightly.

Compared with performing systematic and targeted biopsies on all men, performing this only for men with an elevated Stockholm3 test decreased detection of GG≥2 cancer slightly (RS 0.92; 95% CI 0.88-0.95). Limitations include lacking knowledge of true disease prevalence. These findings are stated to provide evidence that strategies combining the blood-based Stockholm3 test and MRI-targeted biopsies can be used to inform biopsy decision making. Radtke et al in Eur Urol. 2017; 72(6): 888-896 report that multiparametric magnetic resonance imaging (mpMRI) is gaining widespread acceptance in prostate cancer (PC) diagnosis and improves significant PC (sPC; Gleason score≥3+4) detection. Decision making based on European Randomised Study of Screening for PC (ERSPC) risk-calculator (RC) parameters may overcome prostate-specific antigen (PSA) limitations. They added pre-biopsy mpMRI to ERSPC-RC parameters and developed risk models (RMs) to predict individual sPC risk for biopsy-naïve men and men after previous biopsy. They retrospectively analyzed clinical parameters of 1159 men who underwent mpMRI prior to MRI/transrectal ultrasound fusion biopsy between 2012 and 2015. Multivariate regression analyses were used to determine significant sPC predictors for RM development. The prediction performance was compared with ERSPC-RCs, RCs refitted on our cohort, Prostate Imaging Reporting and Data System (PI-RADS) v1.0, and ERSPC-RC plus PI-RADSv1.0 using receiver-operating characteristics (ROCs). Discrimination and calibration of the RM, as well as net decision and reduction curve analyses were evaluated based on resampling methods. PSA, prostate volume, digital-rectal examination, and PI-RADS were significant sPC predictors and included in the RMs together with age. The ROC area under the curve of the RM for biopsy-naïve men was comparable with ERSPC-RC3 plus PI-RADSv1.0 (0.83 vs 0.84) but larger compared with ERSPC-RC3 (0.81), refitted RC3 (0.80), and PI-RADS (0.76). For postbiopsy men, the novel RM's discrimination (0.81) was higher, compared with PI-RADS (0.78), ERSPC-RC4 (0.66), refitted RC4 (0.76), and ERSPC-RC4 plus PI-RADSv1.0 (0.78). Both RM benefits exceeded those of ERSPC-RCs and PI-RADS in the decision regarding which patient to receive biopsy and enabled the highest reduction rate of unnecessary biopsies. Limitations include a monocentric design and a lack of PI-RADSv2.0. The novel RMs, incorporating clinical parameters and PI-RADS, is stated to have performed significantly better compared with RMs without PI-RADS and provided measurable benefit in making the decision to biopsy men at a suspicion of PC. For biopsy-naïve patients, both our RM and ERSPC-RC3 plus PI-RADSv1.0 exceeded the prediction performance compared with clinical parameters alone.

Mottet et al in Eur Urol. 2021 February; 79(2):243-262 present a summary of the 2020 version of the European Association of Urology (EAU)-European Association of Nuclear Medicine (EANM)-European Society for Radiotherapy and Oncology (ESTRO)-European Society of Urogenital Radiology (ESUR)-International Society of Geriatric Oncology (SIOG) guidelines on screening, diagnosis, and local treatment of clinically localised prostate cancer (PCa).

The panel performed a literature review of new data, covering the time frame between 2016 and 2020. The guidelines were updated and a strength rating for each recommendation was added based on a systematic review of the evidence. A risk-adapted strategy for identifying men who may develop PCa is advised, generally commencing at 50 yr of age and based on individualised life expectancy. Risk-adapted screening should be offered to men at increased risk from the age of 45 yr and to breast cancer susceptibility gene (BRCA) mutation carriers, who have been confirmed to be at risk of early and aggressive disease (mainly BRAC2), from around 40 yr of age. The use of multiparametric magnetic resonance imaging in order to avoid unnecessary biopsies is recommended. When a biopsy is performed, a combination of targeted and systematic biopsies must be offered. There is currently no place for the routine use of tissue-based biomarkers. Whilst prostate-specific membrane antigen positron emission tomography computed tomography is the most sensitive staging procedure, the lack of outcome benefit remains a major limitation. Active surveillance (AS) should always be discussed with low-risk patients, as well as with selected intermediate-risk patients with favourable International Society of Urological Pathology (ISUP) 2 lesions. Local therapies are addressed, as well as the AS journey and the management of persistent prostate-specific antigen after surgery. A strong recommendation to consider moderate hypofractionation in intermediate-risk patients is provided. Patients with cN1 PCa should be offered a local treatment combined with long-term hormonal treatment. The evidence in the field of diagnosis, staging, and treatment of localised PCa is stated to be evolving rapidly. The 2020 EAU-EANM-ESTRO-ESUR-SIOG guidelines on PCa are stated to summarise the most recent findings and advice for their use in clinical practice. These PCa guidelines reflect the multidisciplinary nature of PCa management.

### SUMMARY OF THE INVENTION

The use of a variety of tools including risk calculators (inputting existing clinical data), mpMRI, or novel biomarkers to support biopsy decision in subjects with PSA in the grey zone range is recommended. However, the results of biomarker tests, clinical patient information and mpMRI data are stand-alone and sometimes contradictory. They can leave the physician in uncertainty since they are not integrated into a single and conclusive result (see FIG. 1).

There is a clear unmet need for combining the complete patient information for a comprehensive PCa risk assessment to achieve the best possible management along the patient journey. Such a solution must allow seamless integration of all relevant data to achieve a high specificity and sensitivity for the detection of csPCa to select patients for biopsy and subsequent treatment. The negative predictive value of the combined risk score needs to be high to safely rule-out cancer in men to give them peace of mind if the cancer risk is low. Testing must be non-invasive, and results need to be presented in an intuitive and actionable graphical user interface (GUI).

As will be evidenced further below, the integration of Proclarix, mpMRI information and prostate volume into a dedicated biopsy decision algorithm, here named Prosgard, significantly outperforms PSAD, mpMRI alone and the RC. This provides strong support for the use of the proposed process in routine PCa diagnostic practice to improve the biopsy decision algorithm in men with a positive mpMRI (PI-RADS 3-5) outcome and especially in men with indeterminate mpMRI (PI-RADS 3) outcome to reduce the number of unneeded biopsies.

WO 2018/011212, and the corresponding commercial product Proclarix (Proteomedix, Switzerland) is a new blood-based CE-marked test that calculates the PCa risk score after measuring thrombospondin-1 (THBS1), cathepsin D (CTSD), total PSA (tPSA), free PSA (fPSA) in serum, and age. The system was originally developed for use in men with a PSA of 2-10 ng/ml, a prostate volume of ≥35 cm³ and a normal, non-cancer suspicious DRE.

The THBS1 and CTSD glycoproteins were identified, through a targeted proteomic strategy for biomarker discovery, in a PI3K/PTEN cancer pathway model that is involved in the carcinogenesis and progression of PCa. Recently it has been reported that Proclarix can improve csPCa detection by reducing unnecessary prostate biopsies. It was also suggested that the system was more effective than PSAD and the percentage of free PSA among men with positive mpMRI.

To enable a personalized approach for PCa diagnostics along the whole clinical pathway, we propose to integrate multiple diagnostic methods to deliver a unique solution for PCa diagnosis and management (see FIG. 2a).

The new approach (also termed Prosgard) combines the (i) biomarker-based test Proclarix, PI-RADS results from mpMRI together with the patient's prostate volume. A clinical decision support system for visualization termed Cockpit provides a comprehensive tool to guide clinical decisions through integration and visualization of all data and matching with guideline recommendation (see FIG. 2b). A complete patient workflow management platform allows healthcare specialists to register and track patients through the whole prostate cancer diagnostic process and enable a communication among physicians and patients. Moreover, the optimization and compliance with hospital information systems and informatics structures enables a seamless integration and improved workflow to augment productivity and communication in the clinical routine.

A study was initiated to evaluate the diagnostic accuracy of Prosgard in patients with a suspicion of PCa that underwent biopsy of the prostate. Furthermore, the clinical performance of Prosgard was compared with PSAD, mpMRI and RC, respectively.

Generally speaking, the present invention relates to a method for collecting information about the health status of a subject involving the quantitative detection, in serum, plasma or blood of the subject, of the concentration of THBS1, as well as the proportion of free PSA (% fPSA) in combination with data obtained from multi-parametric prostate magnetic resonance imaging data.

Preferably, the multi-parametric prostate magnetic resonance imaging data is used in the form of a PI-RADS evaluation. For the corresponding PI-RADS evaluation reference is made to the corresponding specification as defined in PI-RADS, Prostate Imaging—Reporting and Data System, 2019, Version 2.1 (ACR-ESUR-AdMeTech 2019, DOI: https://doi.org/10.1016/j.eururo.2019.02.033); as for the specifics of this method for the PI-RADS evaluation the disclosure of this reference is expressly included into the specification.

According to a preferred embodiment, the combined assessment involves further combination with a prostate volume parameter.

Preferably, the proposed method involves the quantitative detection, in serum, plasma or blood of the subject, of the concentration of THBS1, the proportion of free PSA (% fPSA), as well as the concentration of at least one protein selected from the group consisting of CTSD, OLFM4, ICAM1. As for the details of the biomarker detection is concerned, specific reference is made to the teaching of WO 2018/011212, the disclosure of which is expressly included into this disclosure.

According to yet another preferred embodiment, the method includes a first step being performed by contacting the subject's serum, plasma or blood, preferably after dilution thereof, with at least one, preferably two, affinity reagent for each protein and detecting whether binding occurs between the respective protein and the at least one affinity reagent and using quantitative readout of the respective protein's concentration, allowing the calculation of the respective concentration in the original serum, plasma or blood, or in case of free PSA its proportion;

a second step of calculating, based on all the protein concentrations as well as the free PSA proportion determined in the first step, a combined score value, wherein preferably after the second step in a third step the risk of a positive biopsy and/or prostate cancer of the subject as based on the biomarkers is determined based on the combined score value as determined in the second step.

Particularly preferably, a combined score value is calculated using the following formula:

$$[ 1 - \frac{1}{1 + {e}^{( {β}_{0} + {β}_{1} * {x}_{1} + … + {β}_{k} * {x}_{k} )}} ] * 100$$

wherein βi are regression coefficients as determined beforehand with an optimization, preferably a maximization of the AUC in a ROC approach, using experimental data, β₀ being the intercept, and wherein xᵢ is
as x₁ a risk score according to claim 5 expressed in the range of 0-1,
as x₂ the prostate volume (expressed in mL), and
as x₃ a PI-RADS score, expressed as integer in the range of 1-5.

Preferably, the parameters in this formula are chosen according to at least one of the following conditions:
β₀ is in the range of (−6)-(−3), preferably in the range of (−5.83)-(−3.47);
and/or β₁ is in the range of 0.01-0.04, preferably in the range of 0.017-0.037;
and/or β₂ is in the range of (−0.03)-(−0.01), preferably in the range of (−0.027)-(−0.010);
and/or β₃ is in the range of 0.9-1.5, preferably in the range of 0.95-0.149.

Typically, for a greater than 90% sensitivity a threshold value of the combined score value of 15-32 or 16.5-31.1 is selected, preferably 21-29 is selected.

Further embodiments of the invention are laid down in the dependent claims.

### BRIEF DESCRIPTION OF THE DRAWINGS

Preferred embodiments of the invention are described in the following with reference to the drawings, which are for the purpose of illustrating the present preferred embodiments of the invention and not for the purpose of limiting the same. In the drawings,

FIG. 1 shows a prostate cancer diagnostic process consisting of interpreting mpMRI, clinical and biomarker data in isolation to decide who needs a biopsy of the prostate;

FIG. 2 shows how in a) the proposed new system, termed “Prosgard”, integrates individual diagnostic components to enhance patient management along the patient journey and achieve the highest accuracy and in b) a cockpit visualization of all integrated data;

FIG. 3 shows patients according to disease status. Percentages of men with clinically significant, insignificant, or no cancer, identified according to PI-RADS v2 scores, are shown. Scores range from 1 to 5, with higher numbers indicating a greater likelihood of clinically significant cancer. PI-RADS v2=Prostate Image Reporting and Data System version 2;

FIG. 4 shows Prosgard risk score correlation with aggressiveness of PCa detected on biopsy. Boxplot shows an increasing Prosgard risk score in the total population (n=562) for more significant cancer based on grade group. Kruskal-Wallis (p<0.001).

FIG. 5 shows Prosgard risk score correlation with PI-RADS v2 scores. A) Boxplot shows an increasing risk score for PI-RADS score 3 and higher and thus clinically significant disease. Kruskal-Wallis (p<0.001). B) Boxplot shows a significantly higher risk score for positive (GG≥2) cases among PI-RADS score 3 (p<0.001).

FIG. 6 shows receiver operating characteristic curves of Prosgard (upper left line), PI-RADS v2 score from mpMRI (middle line) and Proclarix (lower right line) for predicting significant (GG≥2) prostate cancer.

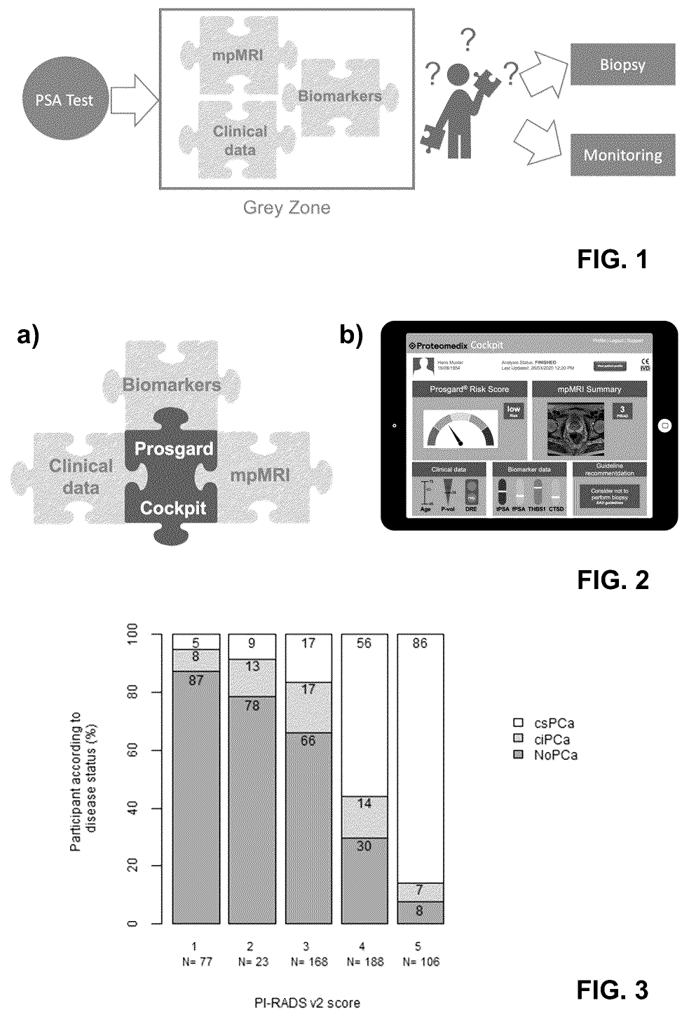

### DESCRIPTION OF PREFERRED EMBODIMENTS

This study is a retrospective single center study, carried out in January 2021 with serum samples and patient data from Vall d'Hebron University Hospital.

### Study Population

Prosgard was assessed retrospectively in a consecutive cohort of 562 men with suspected PCa. The mpMRI sequences were interpreted using the PI-RADS by the local radiologists.

Histopathologic examination of biopsy specimen was performed according to the established practice. CsPCa was defined as ISUP GG≥2 detected on biopsy. A 3T-mpMRI with 2 to 3-core transrectal ultrasound (TRUS) guided biopsies in PI-RADS>3 lesions and/or 12-core TRUS systematic biopsy were carried out from Jan. 15, 2018 to Mar. 20, 2020. Serum samples were obtained before prostate biopsy. Demographic and clinical information was collected in an electronic case record form. This project was approved by the institutional Ethics Committee (PR-AG129/2020).

### Prosgard Testing and Risk Calculation

Frozen serum was stored in the Vall d′Hebron University laboratory at −80° C. (Collection 0003439) until shipment on dry ice to Proteomedix (Zurich-Schlieren, Switzerland) for analysis. Processing of serum samples, the ELISA kit and calculation of the risk score by laboratory technicians were performed blindly before availability to all clinical and biopsy information. THBS1 and CTSD were measured using the CE-marked Proclarix kit (Proteomedix, Zurich-Schlieren, Switzerland) as described before. Serum tPSA and fPSA were reanalyzed for all samples using the Roche Cobas immunoassay system (Roche Diagnostics, Rotkreuz, Switzerland). The values from Proclarix risk score, mpMRI-based PI-RADS score and patient's prostate volume were used to calculate the Prosgard risk score using a novel proprietary algorithm. The cut-off of the Prosgard risk score was set to 25 (95% CI 16.5-31.1).

The combined Prosgard score value is preferably calculated using the following formula:

$$[ 1 - \frac{1}{1 + {e}^{( {β}_{0} + {β}_{1} * {x}_{1} + … + {β}_{k} * {x}_{k} )}} ] * 100$$

wherein βᵢ are the regression coefficients as determined beforehand with an optimization, typically a maximization of the AUC in a ROC approach, using experimental data, β₀ being the intercept, and wherein xᵢ is the Proclarix risk score (expressed in the range of 0-1), the PI-RADS score (expressed as integer in the range of 1-5) and the prostate volume (expressed in mL). The index i therefore in the present situation is 3.

For the calculation of the combined score value the regression coefficients are chosen as follows:

| | Coefficient | Coef. | Coef. | | | |
|---|---|---|---|---|---|---|
| | (βᵢ) | Estimate | Estimate | | | |
| | Estimate | low.95% CI | up.95% CI | Std.Error | z value | p.value |
| Intercept (β₀) | — | — | — | 0.6026627 | −7.722818 | <0.0000001 |
| | 4.6542544 | 5.8354516 | 3.4730572 | | | |
| Proclarix_score | 0.0277756 | 0.0178235 | 0.0377276 | 0.0050777 | 5.470144 | <0.0000001 |
| Volume | — | — | — | 0.0042826 | −4.350624 | 0.0000136 |
| | 0.0186321 | 0.0270258 | 0.0102383 | | | |
| PI-RADS | 1.2268158 | 0.9574398 | 1.4961918 | 0.1374393 | 8.926239 | <0.0000001 |

β₀ in the range of (−6)-(−3), preferably in the range of (−5.83)-(−3.47);
β_{Proclarix} in the range of 0.01-0.04, preferably in the range of 0.017-0.037;
β_{VOLUME} in the range of (−0.03)-(−0.01), preferably in the range of (−0.027)-(−0.010);
β_{PI-RADS} in the range of 0.9-1.5, preferably in the range of 0.95-0.149;

For a greater than 90% sensitivity a threshold value of the combined score value in the range of 16.5-31.1 is selected, preferably 21-29 or specifically 25 is selected.

### Statistical Analyses

Primary endpoint was the correlation of Prosgard with biopsy outcome and comparison with established procedures. Here we assessed Prosgard's superior reduction of unneeded biopsies compared to PI-RADS, PSAD and RC.

The secondary and tertiary endpoints were the correlation of Prosgard in men with positive (PI-RADS 3-5) and indeterminate (PI-RADS 3) mpMRI respectively and assess the reduction of unneeded biopsies compared to PSAD and RC. The difference in specificities and sensitivities were assessed using the McNemar-test and are presented with the 95%-CI [14]. P-values for differences in NPV and PPV were determined according to Moskowitz and Pepe.

### Study Population

In the total study population of 562 men, 230 (41%) men were diagnosed with csPCa based on mpMRI-guided biopsy. The cohort median age and PSA were 69 (IQR 63-74) and 7.0 (4.9-11.2) ng/ml (Table 1).

TABLE 1
Population characteristics.

| | csPCa (GG ≥ 2) | | | | | |
|---|---|---|---|---|---|---|
| | Absence | | Presence | | All | |
| Patients, n | 332 | | 230 | | 562 | |
| Age, years | 67 | [61-72] | 72 | [67-76] | 69 | [63-74] |
| tPSA, ng/ml | 6.1 | [4.5-9.8] | 8.0 | [5.9-14.2] | 7.0 | [4.9-11.2] |
| fPSA/tPSA ratio, % | 17.2 | [12.5-23.4] | 12.0 | [8.8-17.1] | 15.1 | [10.7-20.6] |
| PSA density | 0.103 | [0.072-0.161] | 0.188 | [0.119-0.335] | 0.131 | [0.081-0.207] |
| Prosgard Risk Score, % | 15.8 | [5.0-36.8] | 73.2 | [46.4-88.3] | 35.0 | [11.4-69.4] |
| Prostate volume, cm³ | 64 | [45-86] | 48 | [35-62] | 55 | [40-76] |

csPCa = clinically significant prostate cancer;
Median [25%, 75%];
GG = grade group

The percentage of no PCa in men with a negative result (PI-RADS 1-2) on mpMRI was high for both participants with a PI-RADS score of 1 (87%) and those with a score of 2 (78%). While csPCa was detected in 5% of men in the former group, 9% of men with a PI-RADS score of 2 were diagnosed with csPCa.

Among men with a positive result (PI-RADS 3-5) on mpMRI the percentage of men with clinically significant cancer was highest among participants with a PI-RADS score of 5 (86%), followed by those with a score of 4 (56%) and those with a score of 3 (17%).

Conversely, the percentage of men without cancer was highest among participants with a PI-RADS score of 3 (66%), followed by those with a score of 4 (30%) and those with a score of 5 (8%) (see FIG. 3).

Prosgard correlation with csPCa (GG≥2)

The sensitivity of Prosgard for predicting csPCa was 92% in the total cohort (n=562). When compared to PSAD at equal sensitivity (corresponding to a calculated cut-off of 0.065 for PSAD), Prosgard had a significantly higher specificity (64% vs. 22% [Difference=42%, p<0.001). The negative predictive value (NPV) of Prosgard was 92% [95% CI, 89-96%] and the positive predictive value (PPV) was 64% [95% CI, 59-69%]. In addition, mpMRI and RC results were compared with Prosgard. When the cut-off of mpMRI and the RC were set to PI-RADS 3 and 3%, respectively, sensitivities of 97% (mpMRI) and 95% (RC) were obtained. The specificities, however, were significantly lower when compared to Prosgard (see Table 2).

The nature of the missed csPCa cases for Prosgard and PSAD at the same sensitivity was performed. A 92% sensitivity translated in missing 18 out of 230 csPCa cases. Eleven GG2, three GG3, three GG4 and one GG5 were missed by Prosgard. In comparison, a PSAD using cutoff of 0.065 resulted in the misdiagnosis of seven GG2, four GG3, five GG4 and two GG5 cases.

TABLE 2
Performance characteristics of Prosgard risk score compared to PSA-density, PI-
RADS (mpMRI) and ERSPC online risk calculator in the total cohort (n = 562).

| | | | | | | | | ERSPC | |
|---|---|---|---|---|---|---|---|---|---|
| | | PSA- | | | PI- | | | risk | |
| | Prosgard | density | p- | Prosgard | RADS | p- | Prosgard | calculator | p- |
| Cut-off | 25% | 0.065 | value | 25% | 3 | value | 25% | 3% | value |
| Sensitivity, % | 92 (89-96) | 92 (89-96) | NA | 92 (89-96) | 97 (95-100) | 0.001 | 92 (89-96) | 95 (93-98) | 0.09 |
| Specificity, % | 64 (59-69) | 22 (18-26) | <0.001 | 64 (59-69) | 28 (24-33) | <0.001 | 64 (59-69) | 33 (28-39) | <0.001 |
| NPV, % | 92 (89-96) | 80 (72-88) | 0.004 | 92 (89-96) | 94 (89-99) | 0.359 | 92 (89-96) | 91 (86-96) | 0.611 |
| PPV, % | 64 (59-69) | 45 (41-50) | <0.001 | 64 (59-69) | 49 (44-53) | <0.001 | 64 (59-69) | 50 (45-54) | <0.001 |
| Missed csPCa | | | | | | | | | |
| GG2 | 11 | 7 | | 11 | 4 | | 11 | 8 | |
| GG3 | 3 | 4 | | 3 | 1 | | 3 | 1 | |
| GG4 | 3 | 5 | | 3 | 1 | | 3 | 2 | |
| GG5 | 1 | 2 | | 1 | 0 | | 1 | 0 | |

Median (95% confidence interval);
csPCa = clinically significant prostate cancer,
NPV = negative predictive value;
PPV = positive predictive value;
mpMRI = multi parametric magnetic resonance imaging,
GG = grade group

Prosgard Association with Cancer Aggressiveness:

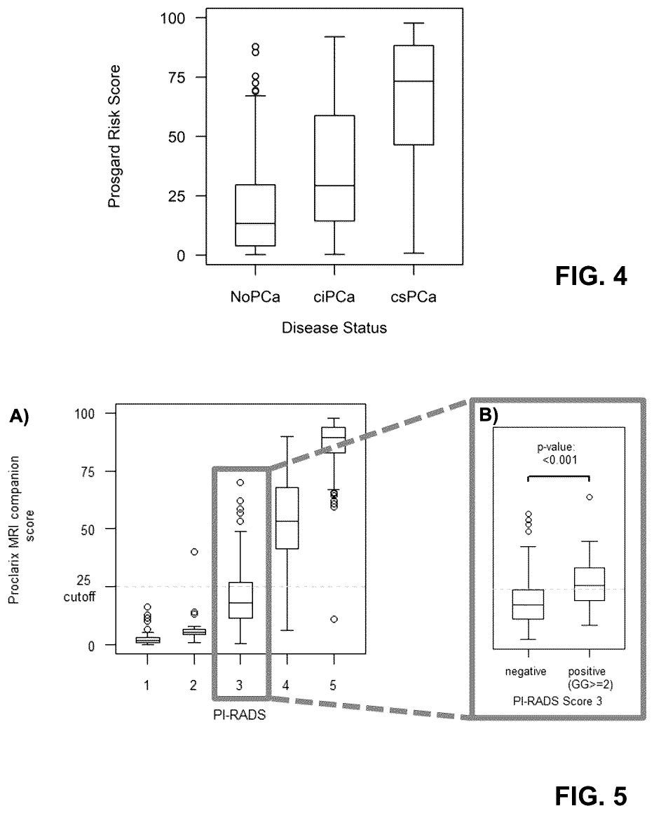

The Prosgard risk score in the total cohort (FIG. 4) significantly correlated with aggressiveness of PCa detected on biopsy (Kruskal-Wallis p<0.001), with an increase of the risk score from noPCa to ciPCa and csPCa subgroups.

The Prosgard risk score showed a significant increase across PI-RADS score groups (FIG. 5A). When specifically looking at the PI-RADS group 3, the Prosgard risk score accurately differentiated csPCa from ciPCa or no PCa (FIG. 5B).

Improving Diagnosis of Positive and Indeterminate mpMRI

A sub-analysis in 462 men with a positive mpMRI (PI-RADS 3-5) outcome resulted in a sensitivity for csPCa of 95% [95% CI, 92-98%], an NPV of 91% [95% CI, 86-96%] and 50% [95% CI, 44-57%] specificity (Table 3).

TABLE 3
Performance characteristics of Prosgard risk score compared to PSA-density and
ERSPC online risk calculator in mpMRI positive (PI-RADS 3-5) men (n = 462).

| | | PSA- | | | ERSPC risk | |
|---|---|---|---|---|---|---|
| | Prosgard | density | | Prosgard | calculator | |
| Cut-off | 25% | 0.065 | p-value | 25% | 3% | p-value |
| Sensitivity, % | 95 (92-98) | 93 (90-97) | 0.491 | 95 (92-98) | 97 (95-99) | 0.134 |
| Specificity, % | 50 (44-57) | 21 (16-27) | <0.001 | 50 (44-57) | 21 (16-27) | <0.001 |
| NPV, % | 91 (86-96) | 77 (67-87) | 0.011 | 91 (86-96) | 90 (82-97) | 0.746 |
| PPV, % | 64 (59-69) | 53 (48-58) | <0.001 | 64 (59-69) | 54 (49-59) | <0.001 |
| Missed csPCa | | | | | | |
| GG2 | 7 | 6 | | 7 | 5 | |
| GG3 | 2 | 3 | | 2 | 0 | |
| GG4 | 2 | 4 | | 2 | 1 | |
| GG5 | 1 | 2 | | 1 | 0 | |

Median (95% confidence interval);
csPCa = clinically significant prostate cancer,
NPV = negative predictive value;
PPV = positive predictive value;
mpMRI = multi parametric magnetic resonance imaging,
GG = grade group

168 (30%) men in this cohort had an indeterminate mpMRI (PI-RADS 3) outcome of whom 28 (17%) had csPCa. When tested in these men, a sensitivity for csPCa of 61% [95% CI, 43-79%], an NPV of 91% [95% CI, 85-96%] and 76% [95% CI, 69-83%] specificity was obtained for Prosgard risk score (Table 4).

TABLE 4
Performance characteristics of Prosgard risk score compared
to PSA-density and ERSPC risk calculator in men with
an indeterminate mpMRI (PI-RADS 3) (n = 168).

| | | PSA- | | | ERSPC risk | |
|---|---|---|---|---|---|---|
| | Prosgard | density | | Prosgard | calculator | |
| Cut-off | 25% | 0.065 | p-value | 25% | 3% | p-value |
| Sensitivity, % | 61 (43-79) | 86 (73-99) | 0.02 | 61 (43-79) | 86 (73-99) | 0.052 |
| Specificity, % | 76 (69-83) | 20 (13-27) | <0.001 | 76 (69-83) | 35 (27-43) | <0.001 |
| NPV, % | 91 (85-96) | 88 (76-99) | 0.558 | 91 (85-96) | 93 (85-100) | 0.649 |
| PPV, % | 33 (20-46) | 18 (11-24) | <0.001 | 33 (20-46) | 21 (13-28) | 0.003 |
| Missed csPCa | | | | | | |
| GG2 | 6 | 3 | | 6 | 4 | |
| GG3 | 2 | 0 | | 2 | 0 | |
| GG4 | 2 | 1 | | 2 | 0 | |
| GG5 | 1 | 0 | | 1 | 0 | |

Median (95% confidence interval);
csPCa = clinically significant prostate cancer,
NPV = negative predictive value;
PPV = positive predictive value;
mpMRI = multi parametric magnetic resonance imaging,
GG = grade group

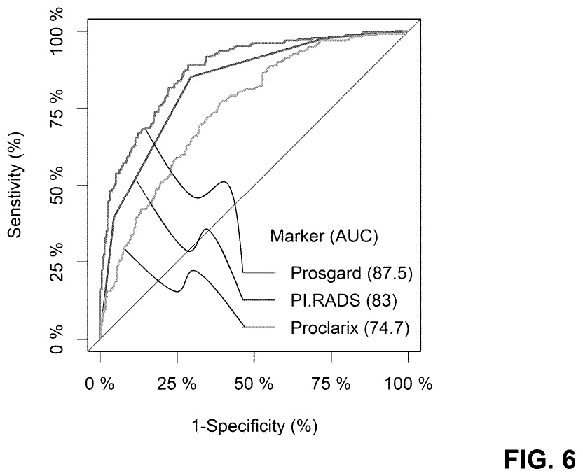

FIG. 6 shows Receiver Operating Characteristic (ROC) curves for Prosgard, PI-RADS and Proclarix. Proclarix discriminated between men with iPCa or no PCa and csPCa bearing patients with an AUC=0.747 (P<0.001; 95% CI=0.707-0.787). PI-RADS resulted in an AUC=0.830 (P<0.001; 95% CI=0.798-0.862). The proposed Prosgard model combining the biomarker-based test Proclarix, PI-RADS results from mpMRI together with the patient's prostate volume resulted in an AUC=0.875 (P<0.001; 95% CI=0.847-0.904). The latter being significantly better than the two former (p<0.001 for both contrasts).

This clinical study was performed to evaluate the clinical performance of Prosgard, an algorithm generating a risk score by integrating the values of Proclarix, mpMRI and prostate volume, for the detection of csPCa.

Results of this study showed that Prosgard overall detects csPCa with high sensitivity and reliably rules out patients with no or insignificant cancer, indicated by a high NPV. Prosgard was compared to PSAD as well as mpMRI and was superior (p<0.001) compared to both determining who can forego a biopsy. The same was true when compared to the results of the RC, a web-based tool routinely used by urologists for biopsy decision making. Prosgard showed superior performance (p<0.001) and could have saved more unneeded biopsies.

Overall, Prosgard was superior to PSAD, mpMRI alone and RC in ruling out unneeded biopsies, with a limited risk of missing csPCa due to its high sensitivity.

One of the most relevant improvement in the early detection of csPCa was the implementation of mpMRI and guided-prostate biopsies. Here we showed that the efficacy of this new strategy can be improved through a more accurate selection of candidates for prostate biopsy using Prosgard.

Even though PI-RADS=3 is defined as mpMRI positive, biopsying all men in this subgroup results in a detection rate of csPCa of not higher than 20% and around 80% of biopsies will be negative. Here the use of PSAD is recommended, especially in clinical settings when magnetic resonance imaging provides an accurate measurement of prostate volume without additional cost or bothers. Importantly, Prosgard could reduce unneeded biopsies by 76% in men with an indeterminate mpMRI (PI-RADS 3) outcome, while achieving a high NPV of 91% and its performance was thus significantly better than PSAD.

1. A method for collecting information about the health status of a subject involving the quantitative detection, in serum, plasma or blood of the subject, of the concentration of THBS1, as well as the proportion of free PSA (% fPSA) in combination with data obtained from multi-parametric prostate magnetic resonance imaging data.

2. A method according to claim 1, wherein the multi-parametric prostate magnetic resonance imaging data is used in the form of a PI-RADS evaluation.

3. A method according to claim 1, wherein it involves further combination with a prostate volume parameter.

4. Method according to claim 1, involving the quantitative detection, in serum, plasma or blood of the subject, of the concentration of THBS1, the proportion of free PSA (% fPSA), as well as the concentration of at least one protein selected from the group consisting of CTSD, OLFM4, ICAM1.

5. Method according to claim 1, wherein the method includes
a first step being performed by contacting the subject's serum, plasma or blood, with at least one or two affinity reagent(s) for each protein and detecting whether binding occurs between the respective protein and the at least one affinity reagent and using quantitative readout of the respective protein's concentration, allowing the calculation of the respective concentration in the original serum, plasma or blood, or in case of free PSA its proportion;
a second step of calculating, based on all the protein concentrations as well as the free PSA proportion determined in the first step, a combined score value.

6. Method according to claim 1, wherein a combined score value is calculated using the following formula:
$$[ 1 - \frac{1}{1 + {e}^{( {β}_{0} + {β}_{1} * {x}_{1} + … + {β}_{k} * {x}_{k} )}} ] * 100$$
wherein βi are regression coefficients as determined beforehand with an optimization using experimental data, β₀ being the intercept, and wherein xᵢ is
as x₁ a risk score expressed in the range of 0-1,
as x₂ the prostate volume (expressed in mL), and
as x₃ a PI-RADS score, expressed as integer in the range of 1-5.

7. Method according to claim 6, wherein
β₀ is in the range of (−6)-(−3);
and/or β₁ is in the range of 0.01-0.04;
and/or β₂ is in the range of (−0.03)-(−0.01);
and/or β₃ is in the range of 0.9-1.5.

8. Method according to claim 6, wherein
β₀ is in the range of (−5.83)-(−3.47);
and/or β₁ is in the range of 0.017-0.037;
and/or β₂ is in the range of (−0.027)-(−0.010);
and/or β₃ is in the range of 0.95-0.149.

9. Method according to claim 6, wherein for a greater than 90% sensitivity a threshold value of the combined score value in the range of 15-32 or 16.5-31.1 is selected, preferably or 21-29 is selected.

10. Method according to claim 1, wherein the quantitative detection, in serum, plasma or blood of the subject method includes
a first step being performed by contacting the subject's serum, plasma or blood, with at least one affinity reagent for each protein and detecting whether binding occurs between the respective protein and the at least one affinity reagent and using quantitative readout of the respective protein's concentration or in case of free PSA its proportion, allowing the calculation of the respective concentration in the original serum, plasma or blood, and wherein in this step either a sandwich enzyme linked immunosorbent assay specific to the respective protein is used, and/or a sandwich bead based antibody assay to the respective protein.

11. Method according to claim 10, wherein the sandwich enzyme linked immunosorbent assay specific to the respective protein and/or the sandwich bead based antibody assay to the respective protein is one obtained by using recombinant proteins of human THBS1, CTSD, ICAM1 and OLFM4, respectively and mouse monoclonal antibodies generated through immunization of mice therewith.

12. Method according to claim 1, wherein the quantitative detection of the respective concentration in the quantitative detection, in serum, plasma or blood of the subject involves the determination of the concentration of such biomarkers relative to an external protein standard, involving the preparation of a reference standard curve by measuring defined concentrations of several, protein standards diluted in the same buffer as for the protein dilution to be measured in the same set of measurements of the samples.

13. Method according to claim 1, wherein the method includes
a first step being performed by contacting the subject's serum, plasma or blood, after dilution thereof, with at least one or two affinity reagent(s) for each protein and detecting whether binding occurs between the respective protein and the at least one affinity reagent and using quantitative readout of the respective protein's concentration, allowing the calculation of the respective concentration in the original serum, plasma or blood, or in case of free PSA its proportion;
a second step of calculating, based on all the protein concentrations as well as the free PSA proportion determined in the first step, a combined score value, wherein after the second step in a third step the risk of a positive biopsy and/or prostate cancer of the subject as based on the biomarkers is determined based on the combined score value as determined in the second step.

14. Method according to claim 1, wherein a combined score value is calculated using the following formula:
$$[ 1 - \frac{1}{1 + {e}^{( {β}_{0} + {β}_{1} * {x}_{1} + … + {β}_{k} * {x}_{k} )}} ] * 100$$
wherein βi are regression coefficients as determined beforehand with a maximization of the AUC in a ROC approach using experimental data, β₀ being the intercept, and wherein xᵢ is
as x₁ a risk score expressed in the range of 0-1,
as x₂ the prostate volume (expressed in mL), and
as x₃ a PI-RADS score, expressed as integer in the range of 1-5.

15. Method according to claim 1, wherein the quantitative detection, in serum, plasma or blood of the subject method includes
a first step being performed by contacting the subject's serum, plasma or blood, after dilution thereof, with at least one affinity reagent for each protein and detecting whether binding occurs between the respective protein and the at least one affinity reagent and using quantitative readout of the respective protein's concentration or in case of free PSA its proportion, allowing the calculation of the respective concentration in the original serum, plasma or blood, and wherein in this step either a sandwich enzyme linked immunosorbent assay specific to the respective protein with visible readout is used, and/or a sandwich bead based antibody assay to the respective protein with fluorescent readout.

16. Method according to claim 10, wherein the sandwich enzyme linked immunosorbent assay specific to the respective protein with visible readout and/or the sandwich bead based antibody assay to the respective protein with fluorescent readout is one obtained by using recombinant proteins of human THBS1, CTSD, ICAM1 and OLFM4, respectively and mouse monoclonal antibodies generated through immunization of mice therewith.

17. Method according to claim 1, wherein the quantitative detection of the respective concentration in the quantitative detection, in serum, plasma or blood of the subject involves the determination of the concentration of such biomarkers relative to an external protein standard, involving the preparation of a reference standard curve by measuring defined concentrations of 5-7 protein standards diluted in the same buffer as for the protein dilution to be measured in the same set of measurements of the samples.
